(19) United States
(12) Patent Application Publication
(10) Pub. No.: US 2009/0298864 A1
(43) Pub. Date: Dec. 3, 2009

(54) Methods for Treating Mild Cognitive Impairment

(75) Inventors: Ottavio V. Vitolo, New York, NY (US); Ottavio Arancio, New York, NY (US); Michael L. Shelanski, Brooklyn, NY (US)

Correspondence Address: WilmerHale/Columbia University, 399 PARK AVENUE, NEW YORK, NY, 10022, US

(73) Assignee: The Trustees of Columbia University in the City of New York, New York, NY (US)

(21) Appl. No.: 11/918,295

(22) PCT Filed: Apr. 7, 2006
(86) PCT No.: PCT/US2006/013163

§ 371 Date: May 27, 2009

Related U.S. Application Data
(60) Provisional application No. 60/670,103, filed on Apr. 11, 2005.

(51) Int. Cl. A61K 31/435; A61K 31/4353; A61K 31/4015; A61P 25/28

(52) U.S. Cl. 514/297; 514/319; 514/424

(57) ABSTRACT

The present invention relates to compounds, compositions, and methods useful for: (i) treating or preventing mild cognitive impairment, or (ii) delaying the progression from mild cognitive impairment to Alzheimer's disease in a subject in need thereof.

This patent application claims the benefit of all patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### 1. FIELD OF THE INVENTION

The present invention relates to the field of compositions and methods useful for the treatment of cognitive disorders.

### 2. BACKGROUND OF THE INVENTION

Mild cognitive impairment is a term generally used to refer to an early, but abnormal, state of cognitive impairment residing in a transitional zone between normal cognitive function and Alzheimer's disease. Although various researchers have used numerous criteria to define cognitive impairment, the generally accepted criteria for mild cognitive impairment are as follows: (i) the person is neither normal nor demented; (ii) evidence of cognitive deterioration shown by a measured or self-reported decline; and (iii) activities of daily life are preserved and complex instrumental functions are intact or minimally impaired.

The underlying causes of cognitive impairment in mild cognitive impairment have not been determined, thus possible methods of treatment are not easily identified. Although it is believed that many mild cognitive impairment patients have neuropathology similar to that of Alzheimer's disease, the fact that not all mild cognitive impairment patients develop Alzheimer's disease suggests that the pathophysiology of mild cognitive impairment differs from that of Alzheimer's disease.

Phosphodiesterase inhibitors are effective as anti-dementia agents and it has recently been shown that brief treatment of a mouse model of Alzheimer's disease with rolipram, a phosphodiesterase IV inhibitor, improves memory in both long-term potential and contextual learning—both measurements of brain function. Despite the effectiveness of such drugs against Alzheimer's disease, it is yet to be determined whether a similar treatment approach would prove successful in patients suffering from mild cognitive impairment.

Thus, there remains a need in the art for compounds, which are useful for treating mild cognitive impairment in a subject. The present invention addresses this need.

### 3. SUMMARY OF THE INVENTION

The present invention relates to compounds, compositions, and methods useful for: (i) treating or preventing mild cognitive impairment, or (ii) delaying the progression from mild cognitive impairment to Alzheimer's disease in a subject in need thereof.

In one aspect, the present invention provides compounds having the Formula (I):

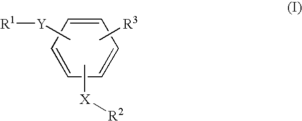

and pharmaceutically acceptable salts thereof
wherein

R² is —H, —C₁-C₆ alkyl, —C₃-C₇ cycloalkyl, —C₃-C₇ cycloalkenyl or -aryl;

R³ is —H, —C₁-C₆ alkyl, —C₃-C₇ cycloalkyl, —C₃-C₇ cycloalkenyl or -aryl;

R³ is —C₃-C₇ cycloalkyl, —C₃-C₇ cycloalkenyl, -aryl or -3- to 7-membered heterocycle;

Y is —O—, —NH— or —S—; and

Z is —O—, —NH— or —S—.

A Compound of Formula (I), or a phosphodiesterase inhibitor (collectively referred to as the “Compounds of the Invention”) are useful for treating or preventing mild cognitive impairment in a subject.

In another aspect, the invention provides compositions comprising an amount of a Compound of the Invention that is effective to treat or prevent mild cognitive impairment, and a physiologically acceptable carrier or vehicle. The compositions are useful for: (i) treating or preventing mild cognitive impairment, or (ii) delaying the progression from mild cognitive impairment to Alzheimer's disease in a subject.

In another aspect, the invention provides methods for: (i) treating or preventing mild cognitive impairment, or (ii) delaying the progression from mild cognitive impairment to Alzheimer's disease, comprising administering to a subject in need thereof an amount of a Compound of the Invention that is effective to treat or prevent mild cognitive impairment or delay the progression from mild cognitive impairment to Alzheimer's disease in a subject in need thereof.

In still another aspect, the invention provides methods for: (i) treating or preventing mild cognitive impairment, or (ii) delaying the progression from mild cognitive impairment to Alzheimer's disease, comprising administering to a subject in need thereof an amount of a phosphodiesterase inhibitor that is effective to treat or prevent mild cognitive impairment or delay the progression from mild cognitive impairment to Alzheimer's disease in a subject in need thereof.

The present invention may be understood more fully by reference to the following detailed description and illustrative examples, which are intended to exemplify non-limiting embodiments of the invention.

### 4. BRIEF DESCRIPTION OF THE DRAWINGS

FIG. 1 shows that administration of rolipram to an APP mouse model of mild cognitive impairment resulted in improved contextual fear learning compared to control mice. The bars of the graph, from left to right represent APP mice treated with vehicle, APP mice treated with rolipram (0.3 mg/kg), control mice treated with vehicle, and control mice treated with rolipram (0.3 mg/kg). The Y-axis represents the percentage of mice in each group showing a “freezing” response to anticipated electrical shock stimuli.

### 5. DETAILED DESCRIPTION OF THE INVENTION

### 5.1 Definitions and Abbreviations

The terms used herein having following meaning:

The term “—C₁-C₆ alkyl” as used herein, refers to a straight chain or branched non-cyclic saturated hydrocarbon having from 1 to 6 carbon atoms, wherein one of the hydrocarbon's hydrogen atoms has been replaced with a single bond. Representative straight chain —C₁-C₆ alkyls include -methyl, -ethyl, -n-propyl, -n-butyl, -n-pentyl, and -n-hexyl. Representative branched —C₁-C₆ alkyls include -isopropyl, -sec-butyl, -isobutyl, -tert-butyl, -isopentyl, -neopentyl, 1-methylbutyl, 2-methylbutyl, 3-methylbutyl, 1,1-dimethylpropyl, 1,2-dimethylpropyl, 1-methylpentyl, 2-methylpentyl, 3-methylpentyl, 4-methylpentyl, 1-ethylbutyl, 2-ethylbutyl, 3-ethylbutyl, 1,1-dimethylbutyl, 1,2-dimethylbutyl, 1,3-dimethylbutyl, 2,2-dimethylbutyl, 2,3-dimethylbutyl, 3,3-dimethylbutyl, -isopropyl, -sec-butyl, -isobutyl, -neohexyl, -isohexyl, and the like. In one embodiment, the C₁-C₆ alkyl is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl.

The term “aryl” as used herein refers to a phenyl group, a biphenyl group, biphenylene group, anthracene group, fulvene group, phenanthrene group or a naphthyl group. In one embodiment, the aryl group is substituted with one or more of the following groups is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl.

The term “C₃-C₇ cycloalkyl” as used herein is a 3-, 4-, 5-, 6- or 7-membered saturated non-aromatic monocyclic cycloalkyl ring. Representative C₃-C₇ monocyclic cycloalkyl groups include, but are not limited to, cyclopropyl, cyclobutyl, cyclopentyl, cyclohexyl and cycloheptyl. In one embodiment, the aryl group is substituted with one or more of the following groups is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl.

The term “C₃-C₇ cycloalkenyl” as used herein is a 3-, 4-, 5-, 6- or 7-membered non-aromatic monocyclic carbocyclic ring having at least one endocyclic double bond, but which is not aromatic. It is to be understood that when any two groups, together with the carbon atom to which they are attached form a C₃-C₇ monocyclic cycloalkenyl group, the carbon atom to which the two groups are attached remain tetravalent. Representative C₃-C₇ monocyclic cycloalkenyl groups include, but are not limited to, cyclopropenyl, cyclobutenyl, 1,3-cyclobutadienyl, cyclopentenyl, 1,3-cyclopentadienyl, cyclohexenyl, 1,3-cyclohexadienyl, cycloheptenyl, 1,3-cycloheptadienyl, 1,4-cycloheptadienyl and -1,3,5-cycloheptatrienyl. In one embodiment, the aryl group is substituted with one or more of the following groups is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl.

The term “halo” as used herein, refers to —F, —Cl, —Br, or —I.

The term “3- to 7-membered heterocycle” refers to: (i) a 3- or 4-membered non-aromatic monocyclic cycloalkyl in which 1 of the ring carbon atoms has been replaced with a N, O or S atom; (ii) a 5-, 6-, or 7-membered aromatic or non-aromatic monocyclic cycloalkyl in which 1-4 of the ring carbon atoms have been independently replaced with a N, O or S atom. The term 3- to 7-membered heterocycle also encompasses any heterocycles described by (i) or (ii) which are fused to a benzene ring, or in which any one of the ring carbon atoms comprises a carbonyl group, such as in lactam and lactone ring systems. The non-aromatic 3- to 7-membered heterocycles can be attached via a ring nitrogen, sulfur, or carbon atom. The aromatic 3- to 7-membered heterocycles are attached via a ring carbon atom. Representative examples of a 3- to 7-membered heterocycle group include, but are not limited to, dihydrofuran-2-one, dihydrofuranyl, furanyl, benzofuranyl, furazanyl, imidazolidinyl, imidazolinyl, imidazolyl, benzimidazolyl, indazolyl, indolinlyl, indolyl, indolizinyl, isoindolinyl, isothiazolyl, isoxazolyl, benzisoxazolyl, morpholinyl, oxadiazolyl, oxazolidinyl, oxazolyl, benzoxazolyl, oxazolidinyl, pyrimidinyl, phenanthridinyl, phenanthrolinyl, piperazinyl, piperidinyl, pyranyl, benzopyranyl, pyrazinyl, pyrazolidinyl, pyrazolinyl, pyrazolyl, pyridazinyl, pyridooxazole, pyridoimidazole, pyridothiazole, pyridyl, pyrimidinyl, pyrrolidinyl, pyrrolinyl, quinolinyl, isoquinolinyl, quinoxalinyl, phthalazinyl, cinnolinyl, quinolizinyl, quinazolinyl, quinuclidinyl, tetrahydrofuranyl, thiadiazinyl, thiadiazolyl, thiazolyl, benzthiazolyl, thienyl, thienothiazolyl, thienooxazolyl, thienoimidazolyl, thiomorpholinyl, thiophenyl, benzothiphenyl, triazinyl, and triazolyl. In one embodiment, the 3- to 7-membered heterocycle group is substituted with one or more of the following groups: is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl.

A “subject” is a mammal, e.g., a human, mouse, rat, guinea pig, dog, cat, horse, cow, pig, or non-human primate, such as a monkey, chimpanzee, baboon or rhesus. In one embodiment, the subject is a human.

Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, butyrate, calcium edetate, camphorsulfonate, camsylate, carbonate, citrate, clavulariate, dihydrochloride, edetate, edisylate, estolate, esylate, fiunarate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A hydrate is another example of a pharmaceutically acceptable salt.

An “effective amount” is an amount of a Compound of the Invention, or an amount of another prophylactic or therapeutic agent, that is effective for: (i) treating or preventing mild cognitive impairment, or (ii) delaying the progression from mild cognitive impairment to Alzheimer's disease.

The term “short-term memory” as used herein, refers to a subject's memory over a time period ranging from about 1 minute to about 24 hours.

The term “long-term memory” as used herein, refers to a subject's memory over a time period that is greater than about 24 hours.

Some chemical structures herein are depicted using bold and dashed lines to represent chemical bonds. These bold and dashed lines depict absolute stereochemistry.

When a first group is “substituted with one or more” second groups, each of one or more of the first group's hydrogen atoms is replaced with a second group. In one embodiment each carbon atom of a first group is independently substituted with one or two second groups. In another embodiment each carbon atom of a first group is independently substituted with only one second group.

### 5.2 The Compounds of the Invention

Compounds of Formula (I) or phosphodiesterase inhibitors (collectively referred to as the “Compounds of the Invention”) are useful for: (i) treating or preventing mild cognitive impairment, or (ii) delaying the progression from mild cognitive impairment to Alzheimer's disease.

### 5.2.1 The Compounds of Formula (I)

As stated above, the present invention encompasses compounds having the Formula (I):

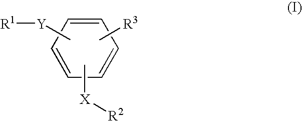

and pharmaceutically acceptable salts thereof, wherein R¹, R², R³, Y and Z are as defined above for the Compounds of Formula (I).

In one embodiment, R¹ is —H.

In another embodiment, R¹ is —C₁-C₆ alkyl.

In still another embodiment, R¹ is —C₃-C₇ cycloalkyl.

In yet another embodiment, R¹ is —C₃-C₇ cycloalkenyl.

In a further embodiment, R¹ is -aryl.

In one embodiment, R¹ is —H.

In another embodiment, R² is —C₁-C₆ alkyl.

In still another embodiment, R² is —C₃-C₇ cycloalkyl.

In yet another embodiment, R² is —C₃-C₇ cycloalkenyl.

In a further embodiment, R² is -aryl.

In one embodiment, R³ is aryl.

In another embodiment, R³ is —C₃-C₇ cycloalkyl.

In yet another embodiment, R³ is —C₃-C₇ cycloalkenyl.

In still another embodiment, R³ is -3- to 7-membered heterocycle.

In one embodiment, Y is —O—.

In another embodiment, Y is —NH—.

In still another embodiment, Y is —S—.

In one embodiment, Z is —O—.

In another embodiment, Z is —NH—.

In still another embodiment, Z is —S—.

Compounds of Formula (I) are useful in the present methods for: (i) treating or preventing mild cognitive impairment, or (ii) delaying the progression from mild cognitive impairment to Alzheimer's disease.

In one embodiment, the present invention provides a method for treating mild cognitive impairment, the method comprising administering to a subject in need thereof an effective amount of a Compound of Formula (I).

In another embodiment, the present invention provides a method for delaying the progression from mild cognitive impairment to Alzheimer's disease, the method comprising administering to a subject in need thereof an effective amount of a Compound of Formula (I).

In one embodiment, the compounds of formula (I) have the formula (Ia):

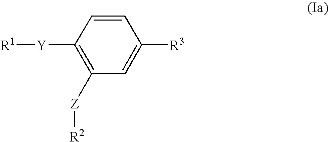

and pharmaceutically acceptable salts thereof, wherein R¹, R², R³, Y and Z are as defined above for the Compounds of Formula (I).

Illustrative Compounds of Formula (I) and Formula (Ia) include:

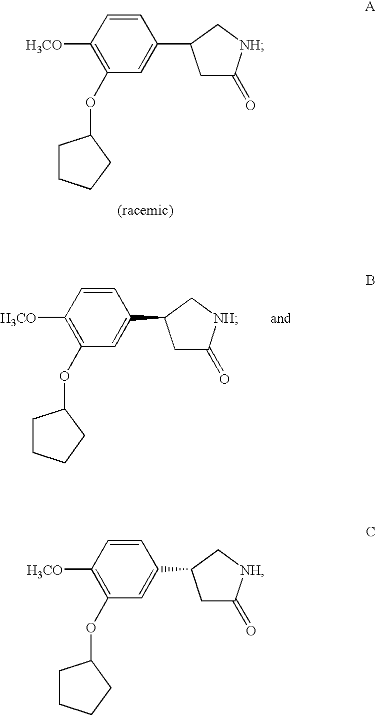

and pharmaceutically acceptable salts thereof.

It is possible for the Compounds of Formula (I) to have one or more chiral centers and as such the Compound of Formula (I) can exist in various stereoisomeric forms. Accordingly, Formula (I), although not depicting specific stereoisomers of the compound of Formula (I), is understood to encompass all possible stereoisomers.

### 5.2.1.1 Methods for Making the Compounds of Formula (I)

The Compounds of Formula (I) may be commercially available, or alternatively, it will be apparent to one of skill in the art of organic synthesis how to choose the proper starting materials and reagents and prepare the Compounds of Formula (I) using the methods disclosed in U.S. Pat. No. 5,591,776 to Cavalla et al.

### 5.2.2 Phosphodiesterase Inhibitors

Phosphodiesterase inhibitors are useful in the present methods for: (i) treating or preventing mild cognitive impairment, or (ii) delaying the progression from mild cognitive impairment to Alzheimer's disease.

In one embodiment, the present invention provides a method for treating mild cognitive impairment, the method comprising administering to a subject in need thereof an effective amount of a phosphodiesterase inhibitor.

In another embodiment, the present invention provides a method for delaying the progression from mild cognitive impairment to Alzheimer's disease, the method comprising administering to a subject in need thereof an effective amount of a phosphodiesterase inhibitor.

In one embodiment, the phosphodiesterase inhibitor is a phosphodiesterase II inhibitor.

In another embodiment, the phosphodiesterase inhibitor is a phosphodiesterase III inhibitor.

In still another embodiment, the phosphodiesterase inhibitor is a phosphodiesterase IV inhibitor.

In yet another embodiment, the phosphodiesterase inhibitor is a phosphodiesterase V inhibitor.

In a further embodiment, the phosphodiesterase inhibitor is a phosphodiesterase VI inhibitor.

In another embodiment, the phosphodiesterase inhibitor is a phosphodiesterase VII inhibitor.

In still another embodiment, the phosphodiesterase inhibitor is a phosphodiesterase VIII inhibitor.

In yet another embodiment, the phosphodiesterase inhibitor is a phosphodiesterase IX inhibitor.

In a further embodiment, the phosphodiesterase inhibitor is a phosphodiesterase X inhibitor.

In another embodiment, the phosphodiesterase inhibitor is a phosphodiesterase XI inhibitor.

Illustrative phosphodiesterase IV inhibitors useful in the present methods for: (i) treating or preventing mild cognitive impairment, or (ii) delaying the progression from mild cognitive impairment to Alzheimer's disease, include but are not limited to, cilomilast, piclamilast, tibenelast, rolipram, benafentrine, zardaverine, tolafentrine and phosphodiesterase IV inhibitors described in International Publication Nos. WO03105902, WO05011602, WO04019945, WO04019944, WO04018465, WO04000806, WO03032981, WO02088080, WO02074312, WO02072586, WO02051502, WO9952848, WO9952847, WO9620175, WO9535282 and WO9535282; European Patent Nos. EP1478399, EP1429807, EP1228046, EP1189888, EP1177175, EP1180100 and EP0710109; United States Patent Publication Nos. US2005043343, US2005026886, US2001044441, US2001041739 and US2003104974; and U.S. Pat. No. 6,300,335, U.S. Pat. No. 6,316,472, and U.S. Pat. No. 6,180,650. In one embodiment, the phosphodiesterase IV inhibitor is rolipram. In a specific embodiment, the phosphodiesterase IV inhibitor is (−) rolipram, (+)-rolipram, or (±)-rolipram.

Illustrative phosphodiesterase V inhibitors useful in the present methods for: (i) treating or preventing mild cognitive impairment, or (ii) delaying the progression from mild cognitive impairment to Alzheimer's disease, include but are not limited to, sildenafil, vardenafil, tadalafil, zaprinast, dipyridamole, papaverine and compounds described in International Publication Nos. WO9306104, WO9849166, WO9954333, WO0024745, WO0127112, WO9307149, WO0259126, WO0118004, WO0200660, WO9312095, WO9405661, WO9400453, WO9519978, WO0127113, WO9924433 and WO9307124; European Patent Nos. EP0995750, EP0995751, EP1092718 and EP1092719; and Rotella et al., J Med Chem, 43(7):1257-63 (2000). In one embodiment, the phosphodiesterase V inhibitor is sildenafil, vardenafil or tadalafil.

Illustrative phosphodiesterase X inhibitors useful in the present methods for: (i) treating or preventing mild cognitive impairment, or (ii) delaying the progression from mild cognitive impairment to Alzheimer's disease, include but are not limited to, papaverine.

### 5.3 Uses of the Compounds of the Invention

In accordance with the invention, the Compounds of the Invention are administered to a subject in need of (i) treatment or prevention of mild cognitive impairment, or (ii) delaying the progression from mild cognitive impairment to Alzheimer's disease.

### 5.3.1 Treatment or Prevention of Mild Cognitive Impairment

The Compounds of the Invention can be used to treat or prevent mild cognitive impairment.

The term “mild cognitive impairment” as used herein, refers to a condition which results in a deterioration in the learning capability, attention, concentration, thinking, or use of language of a subject wherein the deterioration is not severe enough to justify a diagnosis of Alzheimer's disease or other form of dementia. Encompassed within the definition of mild cognitive impairment is the condition commonly known as age-related cognitive decline.

In one embodiment, treating mild cognitive impairment involves lessening the rate of deterioration in a subject's learning capability.

In another embodiment, treating mild cognitive impairment involves lessening the rate of deterioration in a subject's attentiveness.

In still another embodiment, treating mild cognitive impairment lessening the rate of a deterioration in a subject's ability to mentally concentrate.

In yet another embodiment, treating mild cognitive impairment lessening the rate of a deterioration in a subject's ability to think.

In a further embodiment, treating mild cognitive impairment involves lessening the rate of deterioration in a subject's ability to use language.

In one embodiment, treating mild cognitive impairment involves the cessation of deterioration in a subject's learning capability.

In another embodiment, treating mild cognitive impairment involves the cessation of deterioration in a subject's attentiveness.

In still another embodiment, treating mild cognitive impairment involves the cessation of deterioration in a subject's ability to mentally concentrate.

In yet another embodiment, treating mild cognitive impairment involves the cessation of deterioration in a subject's ability to think.

In a further embodiment, treating mild cognitive impairment involves the cessation of deterioration in a subject's ability to use language.

In one embodiment, the invention provides a method for treating mild cognitive impairment, the method comprising administering to a subject in need thereof an effective amount of a compound having the formula:

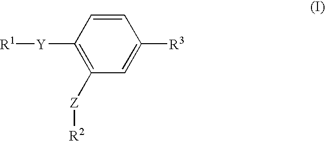

or a pharmaceutically acceptable salt thereof, wherein R¹, R², R³, Y and Z are as defined above for the Compounds of Formula (I).

In another embodiment, the invention provides a method for preventing mild cognitive impairment, the method comprising administering to a subject in need thereof an effective amount of a phosphodiesterase inhibitor.

In one embodiment, the invention provides a method for preventing mild cognitive impairment, the method comprising administering to a subject in need thereof an effective amount of a compound having the formula:

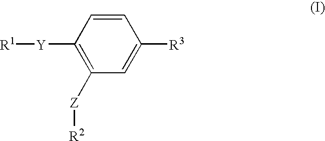

or a pharmaceutically acceptable salt thereof, wherein R¹, R², R³, Y and Z are as defined above for the Compounds of Formula (I).

In another embodiment, the invention provides a method for preventing mild cognitive impairment, the method comprising administering to a subject in need thereof an effective amount of a phosphodiesterase inhibitor.

In one embodiment, the phosphodiesterase inhibitor is a phosphodiesterase II inhibitor.

In another embodiment, the phosphodiesterase inhibitor is a phosphodiesterase III inhibitor.

In still another embodiment, the phosphodiesterase inhibitor is a phosphodiesterase IV inhibitor.

In yet another embodiment, the phosphodiesterase inhibitor is a phosphodiesterase V inhibitor.

In a further embodiment, the phosphodiesterase inhibitor is a phosphodiesterase VI inhibitor.

In another embodiment, the phosphodiesterase inhibitor is a phosphodiesterase VII inhibitor.

In still another embodiment, the phosphodiesterase inhibitor is a phosphodiesterase VIII inhibitor.

In yet another embodiment, the phosphodiesterase inhibitor is a phosphodiesterase IX inhibitor.

In a further embodiment, the phosphodiesterase inhibitor is a phosphodiesterase X inhibitor.

In a specific embodiment, the invention provides a method for treating mild cognitive impairment, the method comprising administering to a subject in need thereof an effective amount of rolipram.

In one embodiment, a subject in need of treatment or prevention of mild cognitive impairment obtains at least one perfect score on the Folstein Mini Mental Status Exam in three administrations of the exam.

In another embodiment, a subject in need of treatment or prevention of mild cognitive impairment receives a rating of 0.5 on the Clinical Dementia Rating Scale.

In still another embodiment, a subject in need of treatment or prevention of mild cognitive impairment scores 1.5 standard deviations or below the age- and education-adjusted normal value on a paragraph recall test.

In one embodiment, the methods of the present invention for treating mild cognitive disorder will result in an improvement in the subject's long-term potentiation.

In another embodiment, the methods of the present invention for treating mild cognitive disorder will result in an improvement in the subject's associative learning capability.

In still another embodiment, the methods of the present invention for treating mild cognitive disorder will result in an improvement in the subject's contextual fear learning capability.

In another embodiment, the methods of the present invention for treating mild cognitive disorder will result in an improvement in the subject's cued fear learning capability.

In yet another embodiment, the methods of the present invention for treating mild cognitive disorder will result in an improvement in the subject's attention span.

In a further embodiment, the methods of the present invention for treating mild cognitive disorder will result in an improvement in the subject's use of language.

In one embodiment, the invention provides a method for improving mild cognitive impairment in an individual afflicted with a cognitive disease or disorder, comprising: (a) administering to the subject a Compound of the Invention for about a week or longer, in an amount effective to stabilize enduring neuronal synaptic function; (b) discontinuing the Compound of the Invention administration for at least 2 weeks or longer; and (c) obtaining an improvement in cognitive function in the afflicted individual. In another embodiment, the improvement comprises an amelioration or correction of one or more deficits in cognition in the subject. In a further embodiment, the Compound of the Invention crosses the blood-brain barrier. In a further embodiment, the Compound of the Invention inhibitor is rolipram. In one embodiment, the Compound of the Invention inhibitor administration occurs daily. In another embodiment, the administering step (a) is for a time period of about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months or about 1 year after administration of the Compound of the Invention. In one embodiment, the discontinuing step (b) is for a time period of about 4 weeks or longer. In another embodiment, the discontinuing step (b) is for a time period of about 6 weeks or longer. In another embodiment, the discontinuing step (b) is for a time period of about 8 weeks or longer. In another embodiment, the discontinuing step (b) is for a time period of about 3 months or longer. In another embodiment, the discontinuing step (b) is for a time period of about 6 months or longer. In another embodiment, the discontinuing step (b) is for a time period of about 9 months or longer. In another embodiment, the discontinuing step (b) is for a time period of about 1 year or longer. In another embodiment, the discontinuing step (b) is for a time period of about 2 years or longer. In another embodiment, the discontinuing step (b) is for a time period of about 3 years or longer. In another embodiment, the discontinuing step (b) is for a time period of about 4 years or longer. In another embodiment, the discontinuing step (b) is for a time period of about 5 years or longer. In another embodiment, the correction of the cognitive deficit involves hippocampal-dependent memory. In another embodiment, the hippocampal-dependent memory comprises short-term memory, such as spatial working memory. In another embodiment, the hippocampal-dependent memory comprises long-term memory, such as reference memory. In another embodiment, the hippocampal-dependent memory comprises associative learning. In a further embodiment, the administering comprises a route selected from intravenous, subcutaneous, intraperitoneal, transdermal, intramuscular, intrathecal, oral, intranasal, intracranial, and any combination thereof. In another embodiment, the stabilizing of an enduring neuronal synaptic function comprises forming new neuronal synapses or consolidating old neuronal synapses. In another embodiment, the subject is afflicted with one or more diseases or disorders selected from Mild Cognitive Impairment, cerebro-vascular dementia, multiple infarct dementia, amyloid angiopathic dementia, cerebro-parenchymatous dementia, senile dementia, Pick's Disease, brain-tumor induced dementia, hydrocephalus-induced dementia, hepatic meningitis-induced dementia, or cerebral trauma-induced dementia. In another embodiment, the subject is afflicted with senile dementia.

The invention also provides for a method for ameliorating or protecting short-term memory in an individual in need thereof, comprising: (a) administering to the subject a Compound of the Invention for about a week or longer, in an amount effective to stabilize enduring neuronal synaptic function; (b) discontinuing the Compound of the Invention administration for at least 2 weeks or longer; and (c) obtaining amelioration or protection of the short-term memory of the subject. In one embodiment, the amelioration or protection comprises correction of one or more deficits in the short-term memory of the subject.

In one embodiment, the short-term memory is spatial working memory.

The invention also provides for a method for ameliorating or protecting long-term memory in an individual in need thereof, comprising: (a) administering to the subject a Compound of the Invention for about a week or longer, in an amount effective to stabilize enduring neuronal synaptic function; (b) discontinuing the Compound of the Invention administration for at least 2 weeks or longer; and (c) obtaining amelioration or protection of the long-term memory of the subject. In one embodiment, the amelioration or protection comprises correction of one or more deficits in the long-term memory of the subject.

In one embodiment, the long-term memory is reference memory.

The invention also provides a method for reversing the inhibitory effects of amyloid beta (Ab) on synaptic plasticity and contextual learning in a subject in need thereof, comprising: (a) administering to the subject a Compound of the Invention for about a week or longer, in an amount effective to stabilize an enduring neuronal synaptic function; (b) discontinuing the Compound of the Invention administration for at least 2 weeks or longer; and (c) achieving a reversal of one or more inhibitory effects of Ab on synaptic plasticity and contextual learning in the subject. In one embodiment, the reversal of Ab inhibition is determined by observing one or more of an increase in long-term memory, an increase in short-term memory, an increase in memory retention, an increase in long-term potentiation (LTP), an increase in cyclic GMP levels, an increase in cyclic AMP levels, or phosphorylation of cyclic AMP response element binding protein (CREB). In another embodiment, the reversal of one or more inhibitory effects of Ab on synaptic plasticity and contextual learning comprises the induction of one or more stabilizing modifications in neurons of the central nervous system. In another embodiment, the one or more stabilizing modifications comprise an alteration of gene expression.

The invention also provides a method for reversing the inhibitory effects of amyloid beta (Ab) on synaptic plasticity and short-term memory in a subject in need thereof, comprising: (a) administering to the subject a Compound of the Invention for about a week or longer, in an amount effective to stabilize an enduring neuronal synaptic function; (b) discontinuing the Compound of the Invention administration for at least 2 weeks or longer; and (c) achieving a reversal of one or more inhibitory effects of Ab on synaptic plasticity and short-term memory in the subject. In one embodiment, the reversal of Ab inhibition is determined by observing one or more of an increase in long-term memory, an increase in short-term memory, an increase in memory retention, an increase in long-term potentiation (LTP), an increase in cyclic GMP levels, an increase in cyclic AMP levels, or phosphorylation of cyclic AMP response element binding protein (CREB). In another embodiment, the reversal of one or more inhibitory effects of Ab on synaptic plasticity and short-term memory comprises the induction of one or more stabilizing modifications in neurons of the central nervous system. In another embodiment, the one or more stabilizing modifications comprise an alteration of gene expression.

The invention also provides a method for reversing the inhibitory effects of amyloid beta (Ab) on synaptic plasticity and long-term memory in a subject in need thereof, comprising: (a) administering to the subject a Compound of the Invention for about a week or longer, in an amount effective to stabilize an enduring neuronal synaptic function; (b) discontinuing the Compound of the Invention administration for at least 2 weeks or longer; and (c) achieving a reversal of one or more inhibitory effects of Ab on synaptic plasticity and long-term memory in the subject. In one embodiment, the reversal of Ab inhibition is determined by observing one or more of an increase in long-term memory, an increase in short-term memory, an increase in memory retention, an increase in long-term potentiation (LTP), an increase in cyclic GMP levels, an increase in cyclic AMP levels, or phosphorylation of cyclic AMP response element binding protein (CREB). In another embodiment, the reversal of one or more inhibitory effects of Ab on synaptic plasticity and long-term memory comprises the induction of one or more stabilizing modifications in neurons of the central nervous system. In another embodiment, the one or more stabilizing modifications comprise an alteration of gene expression.

The present invention also provides for a method for delaying or reducing progression of Mild Cognitive Impairment in a subject, comprising: (a) administering to the subject a Compound of the Invention for about a week or longer, in an amount effective to stabilize enduring neuronal synaptic function; (b) discontinuing the Compound of the Invention administration for at least 2 weeks or longer; and (c) obtaining a delay or reduction in the progression of MCI in the subject. The present invention provides for a treatment method for improving cognitive function in a subject in need thereof, comprising: (a) administering a Compound of the Invention to the subject for at least two weeks prior to suspending administration; (b) suspending the administration for a period; and (c) obtaining an enduring improvement in cognitive function in the treated subject following said suspension period. The invention provides for a method for delaying, reducing, or preventing neuronal damage related to abnormal Ab levels in an individual in need thereof, comprising: (a) administering to the subject a Compound of the Invention for about a week or longer, in an amount effective to stabilize enduring neuronal synaptic function; (b) discontinuing the Compound of the Invention administration for at least 2 weeks or longer; and (c) obtaining a delay, reduction, or prevention of neuronal damage in the subject.

### 5.3.2 Delaying Progression to Alzheimer's Disease

The Compounds of the Invention can be used to delay or prevent the progression from mild cognitive impairment to Alzheimer's disease in a subject.

In one embodiment, the invention provides a method for delaying the progression from mild cognitive impairment to Alzheimer's disease, the method comprising administering to a subject in need thereof an effective amount of a compound having the formula:

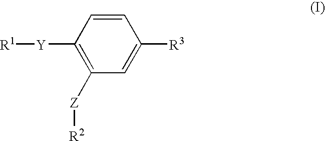

or a pharmaceutically acceptable salt thereof
wherein

R¹ is —H, —C₁-C₆ alkyl, —C₃-C₇ cycloalkyl, —C₃-C₇ cycloalkenyl or -aryl;

R² is —H, —C₁-C₆ alkyl, —C₃-C₇ cycloalkyl, —C₃-C₇ cycloalkenyl or -aryl;

R³ is —C₃-C₇ cycloalkyl, —C₃-C₇ cycloalkenyl, -aryl or -3- to 7-membered heterocycle;

Y is —O—, —NH— or —S—; and

Z is —O—, —NH— or —S—.

In another embodiment, the invention provides a method for delaying the progression from mild cognitive impairment to Alzheimer's disease, the method comprising administering to a subject in need thereof an effective amount of a phosphodiesterase inhibitor.

In still another embodiment, the invention provides a method for delaying the progression from mild cognitive impairment to Alzheimer's disease, the method comprising administering to a subject in need thereof an effective amount of a phosphodiesterase V inhibitor.

In a specific embodiment, the invention provides a method for delaying the progression from mild cognitive impairment to Alzheimer's disease, the method comprising administering to a subject in need thereof an effective amount of rolipram.

In one embodiment, a subject in need of treatment to delay the progression from mild cognitive impairment to Alzheimer's disease bears the apolipoprotein E 64 genotype.

### 5.4 Compositions and Therapeutic Administration of the Compounds of the Invention

The Compounds of the Invention are advantageously useful in veterinary and human medicine. As described above, the Compounds of the Invention are useful for: (i) treating or preventing mild cognitive impairment, or (ii) delaying the progression from mild cognitive impairment to Alzheimer's disease in a subject.

When administered to a subject, the Compounds of the Invention can be administered as a component of a composition that comprises a physiologically acceptable carrier or vehicle. The present compositions, which comprise a Compound of the Invention, can be administered orally or by any other convenient route, for example, by infusion or bolus injection, or by absorption through epithelial or mucocutaneous linings (e.g., oral, rectal, and intestinal mucosa, etc.) and can be administered together with another biologically active agent. Administration can be systemic or local. Various delivery systems are known, e.g., encapsulation in liposomes, microparticles, microcapsules, capsules, etc., and can be administered.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result in the release of the Compounds of the Invention into the bloodstream. The mode of administration is left to the discretion of the practitioner.

In one embodiment, the Compounds of the Invention are administered orally.

In another embodiment, the Compounds of the Invention are administered intravenously.

In still another embodiment, the Compounds of the Invention are administered transdermally.

In other embodiments, it can be desirable to administer the Compounds of the Invention locally. This can be achieved, for example, and not by way of limitation, by local infusion during surgery, by injection, by means of a catheter, by means of a suppository or enema, or by means of an implant, said implant being of a porous, non-porous, or gelatinous material, including membranes, such as sialastic membranes, or fibers.

In certain embodiments, it can be desirable to introduce the Compounds of the Invention into the central nervous system or gastrointestinal tract by any suitable route, including intraventricular, intrathecal, and epidural injection, and enema. Intraventricular injection can be facilitated by an intraventricular catheter, for example, attached to a reservoir, such as an Ommaya reservoir.

Pulmonary administration can also be employed, e.g., by use of an inhaler of nebulizer, and formulation with an aerosolizing agent, or via perfusion in a fluorocarbon or a synthetic pulmonary surfactant.

In another embodiment the Compounds of the Invention can be delivered in a vesicle, in particular a liposome (see Langer, Science 249:1527-1533 (1990) and Liposomes in the Therapy of Infectious Disease and Cancer, pp. 317-327 and 353-365 (1989)).

In yet another embodiment the Compounds of the Invention can be delivered in a controlled-release system or sustained-release system (see, e.g., Goodson, in Medical Applications of Controlled Release, supra, vol. 2, pp. 115-138 (1984)). Other controlled or sustained-release systems discussed in the review by Langer, Science 249:1527-1533 (1990) can be used. In one embodiment a pump can be used (Langer, Science 249:1527-1533 (1990); Sefton, CRC Crit. Ref Biomed. Eng. 14:201 (1987); Buchwald et al, Surgery 88:507 (1980); and Saudek et al., N. Engl. J. Med. 321:574 (1989)). In another embodiment polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al, Science 228:190 (1935); During et al., Ann. Neural. 25:351 (1989); and Howard et al., J. Neurosurg. 71:105 (1989)).

The present compositions can optionally comprise a suitable amount of a physiologically acceptable excipient so as to provide the form for proper administration of a Compound of the Invention to the subject.

Such physiologically acceptable excipients can be liquids, such as water for injection, bactereostatic water for injection, sterile water for injection, and oils, including those of petroleum, subject, vegetable, or synthetic origin, such as peanut oil, soybean oil, mineral oil, sesame oil and the like. The pharmaceutical excipients can be saline, gum acacia; gelatin, starch paste, talc, keratin, colloidal silica, urea and the like. In addition, auxiliary, stabilizing, thickening, lubricating, and coloring agents can be used. In one embodiment the physiologically acceptable excipients are sterile when administered to a subject. Water is a particularly useful excipient when the Compound of the Invention is administered intravenously. Saline solutions and aqueous dextrose and glycerol solutions can also be employed as liquid excipients, particularly for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills; pellets, capsules, capsules containing liquids, powders, sustained-release formulations, suppositories, emulsions, aerosols, sprays, suspensions, or any other form suitable for use. In one embodiment the composition is in the form of a capsule (see e.g. U.S. Pat. No. 5,698,155). Other examples of suitable physiologically acceptable excipients are described in Remington's Pharmaceutical Sciences 1447-1676 (Alfonso R. Gennaro eds., 19th ed. 1995), incorporated herein by reference.

In one embodiment the Compounds of the Invention are formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment the excipients are of pharmaceutical grade.

In one embodiment, when a Compound of the Invention is orally administered, the Compound of the Invention is administered in combination with an additional therapeutic agent that can increase the oral bioavailability of the Compound of the Invention, as described, for example, in U.S. Pat. No. 6,008,222. The additional therapeutic agent may be administered separately from the Compound of the Invention or the additional agent and the Compound of the Invention may be co-administered as part of the same composition.

In another embodiment the Compounds of the Invention can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized-powder or water free concentrate in a hermetically sealed container such as an ampule or sachette indicating the quantity of active agent. Where the Compounds of the Invention are to be administered by infusion, they can be dispensed, for example, with an infusion bottle containing sterile pharmaceutical grade water or saline. Where the Compounds of the Invention are administered by injection, an ampule of sterile water for injection or saline can be provided so that the ingredients can be mixed prior to administration.

The Compounds of the Invention can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,431,922; 5,354;556; and 5,733,556, each of which is incorporated herein by reference. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the Compounds of the Invention of the invention. The invention thus encompasses single unit dosage forms suitable for oral administration such as, but not limited to, tablets, capsules, gelcaps, and caplets that are adapted for controlled- or sustained-release. The invention also encompasses transdermal delivery devices, including but not limited to, a transdermal patch and other devices, such as those described in U.S. Pat. No. 5,633,009.

Controlled- or sustained-release compositions can initially release an amount of a Compound of the Invention that promptly produces the desired diagnostic effect, and gradually and continually release other amounts of the Compound of the Invention to maintain this level of diagnostic effect over an extended period of time. To maintain a constant level of the Compound of the Invention in the body, the Compound of the Invention can be released from the dosage form at a rate that will replace the amount of Compound of the Invention being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions.

The amount of the Compound of the Invention that is effective in the treatment or prevention of mild cognitive impairment can be determined by standard clinical techniques. In addition, in vitro or in vivo assays can optionally be employed to help identify optimal dosage ranges. The precise dose to be employed can also depend on the route of administration, and the seriousness of the condition being treated and can be decided according to the judgment of the practitioner and each subject's circumstances in view of, e.g., published clinical studies. Suitable effective dosage amounts, however, range from about 10 micrograms to about 5 grams about every 4 h, although they are typically about 500 mg or less per every 4 hours. In one embodiment the effective dosage is about 0.01 mg, 0.5 mg, about 1 mg, about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, and about 5.0 g, every 4 hours. Equivalent dosages can be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The effective dosage amounts described herein refer to total amounts administered; that is, if more than one Compound of the Invention is administered, the effective dosage amounts correspond to the total amount administered.

In one embodiment, the Compound of the Invention continues to exert its therapeutic or prophylactic effect for a time period after the administration of the Compound of the Invention. In various embodiments, the Compound of the Invention continues to exert its therapeutic or prophylactic effect for a time period of about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 1 year, about 2 years, about 3 years, about 4 years, or about 5 years after administration of the Compound of the Invention.

Compositions can be prepared according to conventional mixing, granulating or coating methods, respectively, and the present compositions can contain, in one embodiment, from about 0.1% to about 99%; and in another embodiment from about 1% to about 70% of the Compound of the Invention by weight or volume.

The dosage regimen utilizing the Compound of the Invention can be selected in accordance with a variety of factors including type, species, age, weight, sex and medical condition of the subject; the severity of the condition to be treated; the route of administration; the renal or hepatic function of the subject; and the particular Compound of the Invention employed. A person skilled in the art can readily determine the effective amount of the drug useful for: (i) treating or preventing mild cognitive impairment, or (ii) delaying the progression from mild cognitive impairment to Alzheimer's disease.

The Compounds of the Invention can be administered in a single daily dose, or the total daily dosage can be administered in divided doses of two, three or four times daily. Furthermore, the Compounds of the Invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration can be continuous rather than intermittent throughout the dosage regimen. Other illustrative topical preparations include creams, ointments, lotions, aerosol sprays and gels, wherein the concentration of Compound of the Invention ranges from about 0.1% to about 15%, w/w or w/v.

### 5.5 Other Prophylactic/Therapeutic Agents

The present methods for: (i) treating or preventing mild cognitive impairment, or (ii) delaying the progression from mild cognitive impairment to Alzheimer's disease in a subject in need thereof can further comprise administering another prophylactic or therapeutic agent to the subject being administered a Compound of the Invention. In one embodiment the other prophylactic or therapeutic agent is administered in an effective amount. The other prophylactic or therapeutic agent includes, but is not limited to, a cholinesterase inhibitor, an N-methyl D-aspartate antagonist, acetyl-L-carnitine, phosphatidylserine, melatonin, vitamin B6, vitamin B12, vitamin C, vitamin E, or any agent known to be useful in the treatment of Alzheimer's disease.

In one embodiment, the Compound of the Invention can be administered prior to, concurrently with, or after another prophylactic or therapeutic agent, or on the same day, or within 1 hour, 2 hours, 12 hours, 24 hours, 48 hours or 72 hours of each other.

Effective amounts of the other prophylactic or therapeutic agents are well known to those skilled in the art. However, it is well within the skilled artisan's purview to determine the other prophylactic or therapeutic agent's optimal effective amount range. In one embodiment of the invention, where another prophylactic or therapeutic agent is administered to a subject, the effective amount of the Compound of the Invention is less than its effective amount would be where the other prophylactic or therapeutic agent is not administered. In this case, without being bound by theory, it is believed that the Compounds of the Invention and the other prophylactic or therapeutic agent act synergistically to: (i) treat or prevent mild cognitive impairment, or (ii) delay the progression from mild cognitive impairment to Alzheimer's disease.

Cholinesterase inhibitors useful in the methods of the present invention include, but are not limited to, tacrine, galantapine, donezepil and rivastigmine.

N-methyl D-aspartate antagonists useful in the methods of the present invention include, include but are not limited to, memantine.

### 5.6 Kits

The invention encompasses kits that can simplify the administration of a Compound of the Invention to a subject.

A typical kit of the invention comprises a unit dosage form of a Compound of the Invention. In one embodiment the unit dosage form is within a container, which can be sterile, containing a therapeutically effective amount of a Compound of the Invention and a physiologically acceptable carrier or vehicle. The kit can further comprise a label or printed instructions instructing the use of the Compound of the Invention to a subject in need thereof for: (i) treating or preventing mild cognitive impairment, or (ii) delaying the progression from mild cognitive impairment to Alzheimer's disease.

Kits of the invention can further comprise a device that is useful for administering the unit dosage forms. Examples of such a device include, but are not limited to, a syringe, a drip bag, a patch, an inhaler, and an enema bag.

The following examples are set forth to assist in understanding the invention and should not, of course, be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### 5. EXAMPLES

### 5.1 Example 1

### Preparation of Compound a

Compound A can be made according to the procedure set forth in Chang et al., Heterocycles 60:1865-1872 (2003).

### 5.2 Example 2

### Preparation of Compound B

Compound B can be made according to the procedure set forth in Denmitz et al., Molecules 3:107-119 (1998).

### 5.3 Example 3

### Preparation of Compound C

Compound C can be made according to the procedure set forth in Demitz et al., Molecules 3:107-119 (1998).

### 5.4 Example 4

### Effect of Rolipram on Contextual Fear Learning in a Murine Model of Mild Cognitive Impairment

The effect of rolipram on contextual fear learning in APP mice was determined according to the methods set forth in Gong et al., J Clin Invest. 114(11):1624-34 (2004), which are described briefly as follows:

### Step A—Threshold Determination for Response to Foot Shock

Three to five month old single transgenic APP and WT mice were separated into four groups: APP mice treated with vehicle (“APP control group”), APP mice treated with rolipram at 0.3 mg/kg (“APP rolipram group”), WT mice treated with vehicle (“control vehicle group”), and WT mice treated with rolipram at 0.3 mg/kg (“control rolipram group”). 30 minutes after treatment with either vehicle or rolipram, the mice were placed in a conditioning chamber and subjected to a series of electrical shocks to the feet. Animal behavior was evaluated for three types of response to the shocks (flinching, jumping and screaming) and thresholds for various types of responses were determined for each animal

### Step B—Contextual and Cued Fear Conditioning Training

Mice were placed in the conditioning chamber for about 2 minutes, then an auditory tone was given for about 30 seconds. During the last two seconds of the tone, the mice were given a foot shock. The mice were left in the conditioning chamber for about 30 seconds, then were returned to their home cages. “Freezing” behavior, defined as the absence of all movement, was assigned scores using Freezeview software (MED Associates Inc.).

### Step C-Evaluation of Contextual Fear Learning

To evaluate contextual fear learning, freezing was measured for 5 consecutive minutes in the conditioning chamber 24 hours after training.

### Step D—Evaluation of Cued Fear Conditioning

To evaluate cued fear conditioning, after Step C was performed, the mice were placed in a novel context (cage with smooth flat floor and vanilla odorant) for 2 minutes, then exposed to the audio tone described in Step B and freezing was measured.

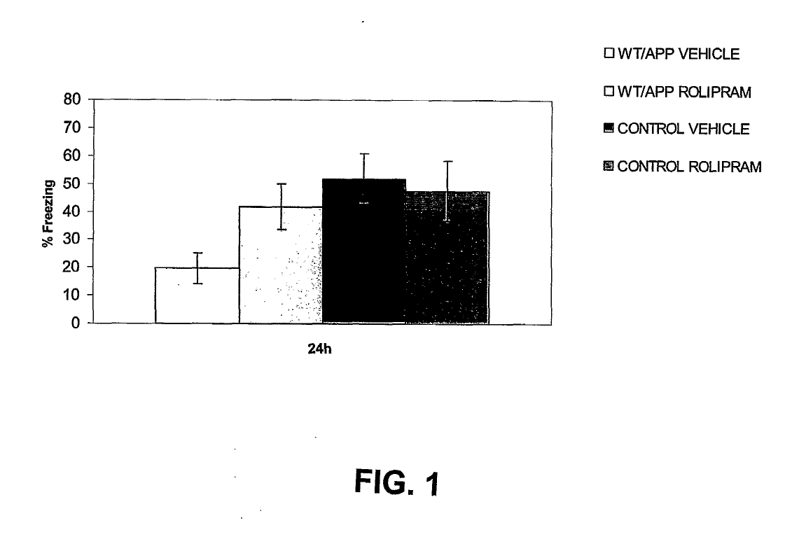

As shown in FIG. 1, APP mice treated with rolipram demonstrated a much higher incidence of freezing in the contextual fear conditioning test than the APP mice treated with vehicle. In contrast, there was no significant difference in the freezing responses of the treated vs. non-treated control mice. These results show that rolipram, an illustrative Compound of the Invention, is useful in increasing contextual fear learning in an animal model of mild cognitive impairment.

What is claimed:

1. A method for treating mild cognitive impairment, the method comprising administering to a subject in need thereof an effective amount of a compound having the formula: 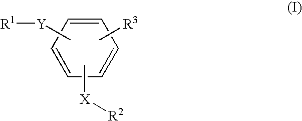
or a pharmaceutically acceptable salt thereof
wherein
R¹ is —H, —C₁-C₆ alkyl, —C₃-C₇ cycloalkyl, —C₃-C₇ cycloalkenyl or -aryl;
R² is —H, —C₁-C₆ alkyl, —C₃-C₇ cycloalkyl, —C₃-C₇ cycloalkenyl or -aryl;
R³ is —C₃-C₇ cycloalkyl, —C₃-C₇ cycloalkenyl, -aryl or -3- to 7-membered heterocycle;
Y is —O—, —NH— or —S—; and
Z is —O—, —NH— or —S—.

2. The method of claim 1, wherein Z is —O—.

3. The method of claim 1, wherein Y is —O—.

4. The method of claim 1, wherein each of Z and Y is —O—.

5. The method of claim 1, wherein R¹ is —C₁-C₆ alkyl.

6. The method of claim 5, wherein R¹ is methyl.

7. The method of claim 1, wherein R² is —C₃-C₇ cycloalkyl.

8. The method of claim 7, wherein R² is cyclopentyl.

9. The method of claim 1, wherein R³ is -3- to 7-membered non-aromatic heterocycle.

10. The method of claim 9, wherein R³ is:

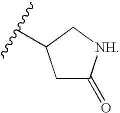

11. The method of claim 4, wherein R¹ is methyl and R² is cyclopentyl.

12. A method for treating mild cognitive impairment, the method comprising administering to a subject in need thereof an effective amount of a phosphodiesterase inhibitor.

13. A method for delaying the progression from mild cognitive impairment to Alzheimer's disease, the method comprising administering to a subject in need thereof an effective amount of a compound having the formula: 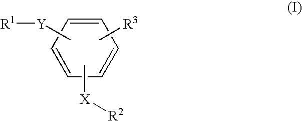
or a pharmaceutically acceptable salt thereof
wherein
R¹ is —H, —C₁-C₆ alkyl, —C₃-C₇ cycloalkyl, —C₃-C₇ cycloalkenyl or -aryl;
R² is —H, —C₁-C₆ alkyl, —C₃-C₇ cycloalkyl, —C₃-C₇ cycloalkenyl or -aryl;
R³ is —C₃-C₇ cycloalkyl, —C₃-C₇ cycloalkenyl, -aryl or -3- to 7-membered heterocycle;
Y is —O—, —NH— or —S—; and
Z is —O—, —NH— or —S—.

14. A method for delaying the progression from mild cognitive impairment to Alzheimer's disease, the method comprising administering to a subject in need thereof an effective amount of a phosphodiesterase inhibitor or a pharmaceutically acceptable salt thereof.

15. The method of claim 1 or 13, wherein the compound of Formula (I) is:

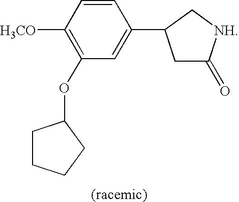

16. The method of claim 1 or 13, wherein the compound of Formula (I) is:

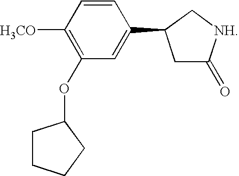

17. The method of claim 1 or 13, wherein the compound of Formula (I) is:

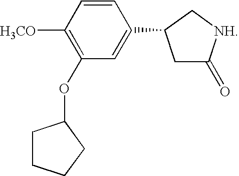

18. The method of claim 13 or 14, wherein the subject bears the apolipoprotein Eε4 genotype.

19. The method of claim 1 or 12, wherein the subject obtains at least one perfect score on the Folstein Mini Mental Status Exam in three administrations of the Exam.

20. The method of claim 1 or 12, wherein the subject receives a rating of 0.5 on the Clinical Dementia Rating Scale.

21. The method of claim 1 or 12, wherein the subject scores 1.5 standard deviations or below the age- and education-adjusted normal value on a paragraph recall test.

22. The method of claim 1 or 12, which results in an improvement in the subject's long-term potentiation.

23. The method of claim 1 or 12, which results in an improvement in the subject's associative learning capability.

24. The method of claim 21, wherein the improvement is in the subject's contextual fear learning capability.

25. The method of claim 1 or 12, which results in an improvement in the subject's attention span.

26. The method of claim 1 or 12, wherein the method results in an improvement in the subject's use of language.

27. The method of claim 1 or 12, wherein the administering comprises oral administration.

28. The method of any one of claims 1, 12, 13 or 14, wherein the subject is a human.

29. The method of claim 1 or 12, further comprising administering to the subject an effective amount of a compound which is a cholinesterase inhibitor or a pharmaceutically acceptable salt thereof.

30. The method of claim 29, wherein the compound which is a cholinesterase inhibitor is tacrine, galantapine, donezepil or rivastigmine.

31. The method of claim 1 or 12, further comprising administering to the subject an effective amount of a compound which is an N-methyl D-aspartate antagonist or a pharmaceutically acceptable salt thereof.

32. The method of claim 31, wherein the compound which is an N-methyl D-aspartate antagonist is memantine.

33. The method of claim 1 or 12, further comprising administering to the subject an effective amount of any of the following compounds or pharmaceutically acceptable salts thereof: acetyl-L-carnitine, phosphatidylserine, melatonin, vitamin B6, vitamin B12, vitamin C or vitamin E.

34. The method of claim 12 or 14, wherein the phosphodiesterase inhibitor is a phosphodiesterase IV inhibitor.

35. The method of claim 34 wherein the phosphodiesterase IV inhibitor is cilomilast, piclamilast, tibenelast, benafentrine, zardaverine or tolafentrine.

36. The method of claim 12 or 14, wherein the phosphodiesterase inhibitor is a phosphodiesterase V inhibitor.

37. The method of claim 36 wherein the phosphodiesterase V inhibitor is sildenafil, vardenafil, tadalafil, zaprinast, dipyridamole or papaverine.

38. The method of claim 12 or 14, wherein the phosphodiesterase inhibitor is a phosphodiesterase X inhibitor.
